(19) United States
(12) Patent Application Publication
(10) Pub. No.: US 2025/0157587 A1
(43) Pub. Date: May 15, 2025

(54) DATA PROCESSING METHOD AND APPARATUS FOR SOLID SYSTEM

(71) Applicant: Beijing Youzhuju Network Technology Co., Ltd., Pinggu District, Beijing (CN)

(72) Inventor: Xiang LI, Beijing (CN)

(21) Appl. No.: 18/723,070

(22) PCT Filed: Mar. 17, 2023
(86) PCT No.: PCT/CN2023/082036

§ 371 Date: Jun. 21, 2024

(30) Foreign Application Priority Data
Mar. 18, 2022 (CN) 202210269495.2

(51) Int. Cl. G16C 10/00; G06N 3/00

(52) U.S. Cl. CPC G16C 10/00; G06N 3/002

(57) ABSTRACT

The present disclosure relates to a data processing method and apparatus for a solid system. The data processing method for a solid system comprises the following steps: performing periodization processing on physical attribute information in a microscopic system state of a solid system; applying the periodized physical attribute information to a specific wave function model; and creating a complex-valued representation on the basis of the output of the specific wave function model.

### CROSS-REFERENCE TO RELATED APPLICATIONS

This application claims priority to and is based on a Chinese application with an application number 202210269495.2 and a filing date of Mar. 18, 2022, the aforementioned application is hereby incorporated by reference in its entirety.

### FIELD OF THE INVENTION

The present disclosure relates to the field of physical technology, and in particular to data processing for solid systems.

### BACKGROUND

Solid state physics belongs to an important branch of physics, and it is a discipline that researches physical properties, microstructure, motion patterns and laws of various particles in solids, and their interrelationships for solids. The object researched by the solid state physics is solids, and the solid state physics aims to interpret macroscopic physical properties of solid materials at a microscopic level. The main theoretical basis in solid state physics research is quantum mechanics. Quantum mechanics describes the operating laws of the microscopic world, and the kernel of quantum mechanics lies in solving a Schrodinger equation of the microscopic system. The Schrodinger equation is a basic equation for quantum mechanics, which reveals the basic laws of motion of matter in the microscopic physical world.

### DISCLOSURE OF THE INVENTION

The Disclosure of The Invention is provided to give a brief overview of concepts, which will be described in detail later in the section Detailed Description of Embodiments. The Disclosure of The Invention is neither intended to identify key or necessary features of the claimed technical solutions, nor is it intended to be used to limit the scope of the claimed technical solutions.

According to some embodiments of the present disclosure, there is provided a data processing method for a solid system, which may include the following steps: performing periodization processing on physical attribute information in a microscopic system state of the solid system; applying the periodized physical attribute information to a specific wave function model to obtain output of the specific wave function model; and creating a complex-valued representation based on the output of the specific wave function model.

According to some other embodiments of the present disclosure, there is provided a data processing apparatus for a solid system, which may include a periodization processing unit configured to perform periodization processing on physical attribute information in a microscopic system state of the solid system; a model application unit configured to apply the periodized physical attribute information to a specific wave function model to obtain output of the specific wave function model; and a complex-valued representation creation unit configured to create a complex-valued representation based on the output of the specific wave function model.

According to some other embodiments of the present disclosure, there is provided an analysis method for a solid system, which may include the following steps: acquiring a complex-valued representation through the data processing method of any embodiment described in the present disclosure, as a wave function value in complex-valued form that reflects physical properties of the solid system and/or satisfies requirements of a wave function of the solid system; and applying the wave function value to solve a specific equation characterizing the microscopic system of the solid system to determine the physics properties of the solid system.

According to some other embodiments of the present disclosure, there is provided an analysis apparatus for a solid system, which may include an acquisition unit configured to acquire a complex-valued representation through the data processing method of any embodiment described in the present disclosure, as a wave function value in complex-valued form that reflects physical properties of the solid system and/or satisfies requirements of a wave function of the solid system; and a solving unit configured to apply the wave function value to solve a specific equation characterizing the microscopic system of the solid system to determine the physics properties of the solid system.

According to some other embodiments of the present disclosure, there is provided an electronic device, which may include: a memory; and a processor coupled with the memory, wherein the processor is configured to, based on instructions stored in the memory, execute the method according to any one of the embodiments of the present disclosure.

According to some other embodiments of the present disclosure, there is provided a computer-readable storage medium having a computer program stored thereon, wherein the program, when executed by a processor, causes the method according to any one of the embodiments of the present disclosure to be implemented.

According to some other embodiments of the present disclosure, there is provided a computer program product including instructions that, when executed by a processor, cause the method according to any one of the embodiments of the present disclosure to be implemented.

According to some other embodiments of the present disclosure, there is provided a computer program including program codes that, when executed by a processor, cause the method according to any one of the embodiments of the present disclosure to be implemented.

Through the following detailed description of exemplary embodiments of the present disclosure with reference to the drawings, other features, aspects, and advantages of the present disclosure will become clear.

### DESCRIPTION OF THE DRAWINGS

Preferred embodiments of the present disclosure are described below with reference to the drawings. The drawings illustrated here are used to provide a further understanding of the present disclosure, and together with the following detailed description, are incorporated in and form a part of the specification, to explain the present disclosure. It should be understood that the drawings in the following description relate to only some embodiments of the present disclosure, and do not constitute a limitation on the present disclosure. In the drawings:

FIGS. 1A and 1B illustrate schematic internal structural diagrams in a solid state system according to embodiments of the present disclosure.

FIG. 2 illustrates the basic concept of physical property research/analysis for a solid system according to embodiments of the present disclosure.

FIG. 3A shows a flowchart of a data processing method for a solid system according to an embodiment of the present disclosure.

FIG. 3B shows a schematic diagram of exemplary data periodic expansion according to an embodiment of the present disclosure.

FIG. 3C shows an overall conceptual diagram of data processing for a solid state system according to an embodiment of the present disclosure.

FIG. 3D shows a block diagram of a data processing apparatus for a solid state system according to an embodiment of the present disclosure.

FIG. 3E shows a flow diagram of an analysis method for a solid system according to an embodiment of the present disclosure.

FIG. 3F shows a block diagram of an analysis apparatus for a solid system according to an embodiment of the present disclosure.

FIG. 4A-4D show renderings of physical property research/analysis for a solid system according to embodiments of the present disclosure.

FIG. 5 shows a block diagram of some embodiments of an electronic device of the present disclosure.

FIG. 6 shows a block diagram of further embodiments of an electronic device of the present disclosure.

It should be understood that, for ease of description, the sizes of various parts shown in the drawings are not necessarily drawn to actual scale. The same or similar reference numerals in the drawings are used to denote the same or similar components. Therefore, once an item has been defined in one of the drawings, it may not be further discussed in the subsequent drawings.

### DETAILED DESCRIPTION OF PREFERRED EMBODIMENTS

The technical solutions in the embodiments of the present disclosure are clearly and completely described below with reference to the drawings of the embodiments of the present disclosure. However, apparently, the embodiments described are merely some embodiments of the present disclosure rather than all the embodiments. The following description of the embodiments is actually merely illustrative, and in no way serves as any limitation to the present disclosure and application or use thereof. It should be understood that the present disclosure may be implemented in various forms and should not be construed as being limited to the embodiments set forth here.

It should be understood that the various steps described in the method implementations of the present disclosure may be performed in different orders, and/or performed in parallel. Furthermore, additional steps may be included and/or the execution of the illustrated steps may be omitted in the method implementations. The scope of the present disclosure is not limited in this respect. Unless specifically stated otherwise, the relative arrangement of components and steps, numerical expressions, and numerical values set forth in these embodiments should be construed as merely exemplary, and do not limit the scope of the present disclosure.

The term “include” and the variations thereof used in the present disclosure are open-ended terms that include at least subsequent elements/features but do not exclude other elements/features, that is, “including but not limited to”. In addition, the term “comprise” and the variations thereof used in the present disclosure are open-ended terms that comprise at least subsequent elements/features but do not exclude other elements/features, that is, “comprising but not limited to”. In the context of the present disclosure, “include” has the same meaning as “comprise”. The term “based on” means “at least partially based on”.

The terms “one embodiment”, “some embodiments”, or “an embodiment” described throughout the specification means that the specific features, structures, or characteristics described in connection with the embodiments are included in at least one embodiment of the present invention. For example, the term “one embodiment” means “at least one embodiment”. The term “another embodiment” means “at least one another embodiment”. The term “some embodiments” means “at least some embodiments”. Moreover, the phrases “in one embodiment”, “in some embodiments”, or “in an embodiment” appearing in various places throughout the specification do not necessarily all refer to the same embodiment, but may also refer to the same embodiment. It should be noted that the modifiers “one” and “a plurality of” mentioned in the present disclosure are illustrative and not restrictive, and those skilled in the art should understand that unless the context clearly indicates otherwise, the modifiers should be understood as “one or more”.

It should be noted that concepts such as “first” and “second” mentioned in the present disclosure are only used to distinguish different apparatuses, modules, or units, and are not used to limit the sequence of functions performed by these apparatuses, modules, or units or interdependence. Unless otherwise specified, concepts such as “first” and “second” are not intended to imply that the objects described in this way must be in a given order in terms of time, space, or ranking, or in any other given order.

In physical research systems, solid systems have always attracted much attention. Solid is a basic form of matter, which can include crystalline solid, amorphous solid, quasicrystal, etc., and its microscopic image includes a bunch of atomic nuclei (about order of 10²³) periodically arranged in a specific way and electrons moving freely therewithin. Since solid systems exist in every aspect of people's daily lives, solid systems have extremely high research value.

Existing calculation methods have respective limitations for solid systems, both in terms of calculation accuracy and scale of simulation systems are greatly restricted. Therefore, it is necessary to find more efficient and accurate methods.

In recent years, machine learning methods have been widely used in physics research. In particular, for molecular systems, a number of powerful neural network wave functions have been proposed. These neural network wave functions provide flexible and powerful wave function forms for researching molecular systems, and have achieved good results. However, solid systems are very different from molecular systems. Specifically, a molecule belongs to a composite system composed of a small number of atoms, while a solid system is composed of macroscopic quantities of atoms arranged periodically. The wave function of a solid system needs to meet periodicity requirements, complex-valued requirements, etc. These requirements prevent existing neural network designs for molecular systems from being effectively applied to the research of solid systems.

Therefore, a main purpose of the present disclosure is to propose an improved and expanded solution that can efficiently and accurately research/analyze solid systems.

The research of solid systems can be carried out by applying quantum mechanics, which usually requires solving a Schrodinger equation that describes microscopic systems in a solid system, such as the movement of microscopic particles. The Schrodinger equation can usually be expressed as HΨ=EΨ, where H is a system Hamiltonian, Ψ is a system wave function, and E is energy. The wave function can characterize/describe the microscopic system state of the solid system, and is also called a probability amplitude function. By acquiring the wave function and solving the Schrodinger equation, the corresponding energy can be obtained, and thereby analysis of the physical properties of the solid system can be implemented.

In view of this, on the one hand, the present disclosure proposes an improved data processing scheme for the solid system. Specifically, considering that the wave function is very critical to research of the solid system, the data processing for the solid system in the present disclosure may essentially be the data processing associated with the wave function of the solid system.

The present disclosure enables optimizing data processing based on the physical properties of the solid system and/or the requirements of the wave function of the solid system to obtain an accurate wave function output characterizing the solid system. In particular, the present disclosure is based on a specific wave function model, such as a conventional wave function model that cannot be directly and effectively applied to the solid system, and based on the physical properties of the solid system and/or the requirements of the wave function of the solid system, data related to the wave function (for example, input and output data of a specific wave function model) are processed to further reflect the physical properties of the solid system and meet the requirements of the wave function of the solid system on the basis of the conventional wave function, and accurate wave function output suitable for the solid system can be obtained in a cost-effective manner. In this way, although a conventional wave function model, such as the neural network model for the molecular system, may not be able to effectively reflect the physical properties of solid system and/or meet the requirements of wave function of the solid system, including but not limited to periodicity and complex-valued characteristics, etc. of the solid system and wave functions of the solid system, the solution of the present disclosure can naturally apply conventional wave function models to solid systems while maintaining their respective accuracy, thereby accurately obtaining wave function output that characterizes the solid system in a cost-effective manner, without the need to refit and construct a wave function specially adapted to the solid system, such as a wave function that meets the periodicity and complex value requirements.

It should be noted that in the present disclosure, performing the data processing related to the wave function based on the physical properties of the solid system and/or the requirements of the wave function of the solid system can be considered to a certain extent equivalent to construction/fitting a wave function suitable for the solid system, for example, a wave function that reflects the physical properties of solid system and/or meets the requirements of wave function of the solid system. In particular, for input data, the output obtained by the data processing of the present disclosure is just like the output obtained by inputting the input data into a solid system wave function that reflects the physical properties of solid system and/or meets the requirements of wave function of the solid system.

On the other hand, the present disclosure proposes improved solid system research/analysis. Specifically, based on the wave function output characterizing the solid system obtained by the data processing method of any embodiment of the present disclosure, it can solve a Schrodinger equation for the solid system, to obtain more accurate solution results, and thereby obtain more accurate analysis of physical properties for the solid system.

The embodiments of the present disclosure will be described in detail below with reference to the accompanying drawings, but the present disclosure is not limited to these specific embodiments. The following specific embodiments can be combined with each other, and the same or similar concepts or processes may not be described again in some embodiments. Furthermore, in one or more embodiments, specific features, structures or characteristics may be combined in any suitable manner that will be apparent to one of ordinary skilled in the art from this disclosure.

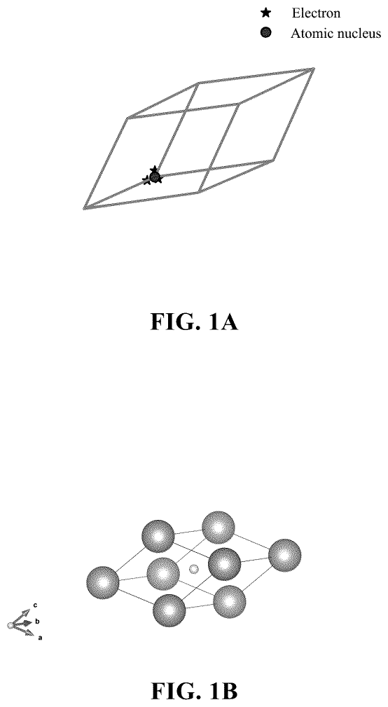

A solid system may consist of periodically arranged atomic nuclei and electrons moving freely therewithin. FIG. 1A shows an atomic nucleus and electrons in an exemplary partial solid system. Structurally, a solid system can be composed of smallest repetitive cell(s). The smallest repetitive cell may refers to the smallest cell in the solid system that can be periodically arranged to cover/compose the entire solid system, and can be composed of a specific number of atomic nuclei, the smallest repetitive cell can be arranged into various appropriate forms, such as cube, cuboid, etc. The arrangement of the smallest repetitive units in a solid system can be indicated by vectors, for example, a normal lattice vector may refer to a vector that describes the periodic arrangement manner of atomic nuclei in the solid system. The smallest repetitive cells can be arranged according to the normal lattice vector to spread over the whole space. FIG. 1B shows an illustration of a smallest repetitive cell in a solid system according to an embodiment of the present disclosure, where spheres represent atomic nuclei in the solid and arrows a, b, c represent normal lattice vectors in the solid. It should be noted that the normal lattice vectors may be vectors that are orthogonal to each other, or vectors that are non-orthogonal, which may depend, for example, on the arrangement of the smallest repetitive cells, which will not be specifically defined in the present disclosure.

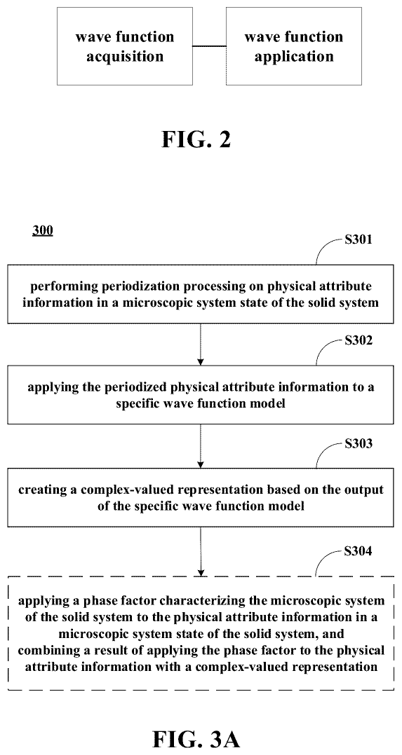

A general conceptual diagram of physical property research/analysis for a solid system according to embodiments of the present disclosure is schematically illustrated in FIG. 2. The physical properties of a solid system can be any appropriate solid physical attributes, such as energy related attributes/indications, etc. As a general idea, for analyzing/researching the solid system, such as determining the physical properties/energy distribution of the solid system, it usually needs to solve a Schrodinger equation that characterizes the microscopic system of the solid system, while the wave function describing the microscopic system state of the solid system is critical. Therefore, through accurately acquiring a wave function that characterizes a solid system, especially the microscopic system state of the solid system, and applying the acquired wave function for equation solving, the physical properties of the solid system can be accurately determined.

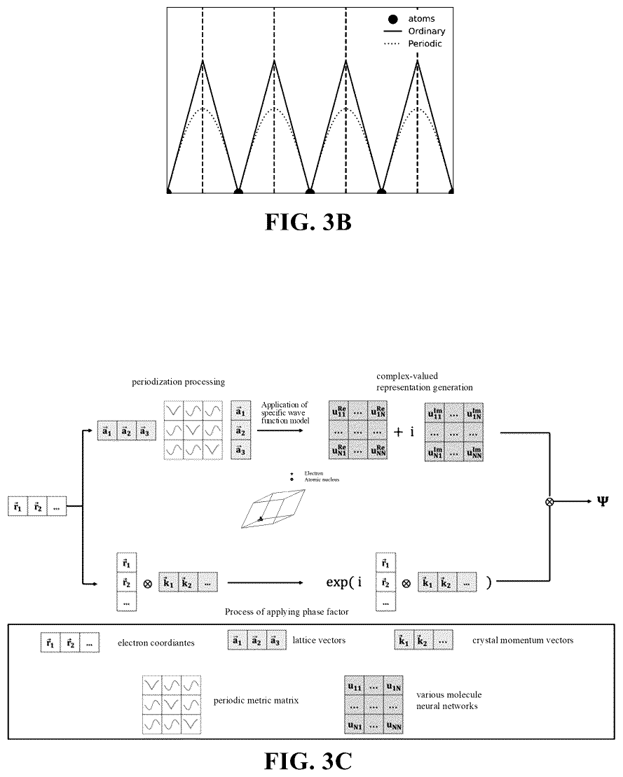

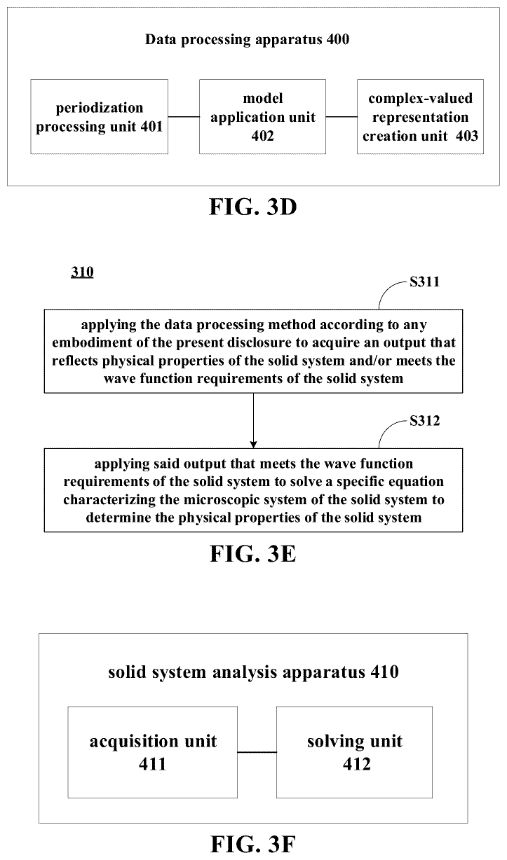

FIG. 3A shows a flowchart of a data processing method for a solid system according to an embodiment of the present disclosure. In the context of the present disclosure, data processing for the solid systems refers in particular to data processing associated with the microscopic state of the solid system, in particular associated with an appropriate function, such as wave function, capable of characterizing the microscopic system state of the solid system, which may include, for example, but not limited to calculation, fitting, etc. of data/numerical values/information. In this disclosure, microscopic refers particularly to an atomic size scale.

In method 300, in step S301, performing periodization processing on physical attribute information in a microscopic system state of the solid system; in step S302, applying the periodized physical attribute information to a specific wave function model to obtain output of the specific wave function model; and in step S303, creating a complex-valued representation based on the output of the specific wave function model.

In some embodiments of the present disclosure, the physical attribute information in the microscopic system state of the solid system may refer to information related to the physical attributes in the microscopic system state of the solid system, for example, information related to states/attributes of electrons in the solid system, including but not limited to information related to the spatial distribution of electrons in the solid system. The information related to the spatial distribution of electrons may include or be based on the spatial coordinates (such as three-dimensional spatial coordinates, which may be in vector form), spatial distance, etc. of electrons.

In some embodiments of the present disclosure, the wave function is a function that describes the microscopic system state, particularly a wave function that describes electron states. Its input can be information about states/attributes of the electrons, such as spatial coordinates of the electrons, and the module square of the output is proportional to the probabilities of the electrons appearing there. In the present disclosure, the wave function can be determined in various appropriate ways, in particular, it can be determined by deep learning, neural network, deep neural network, etc., and can be calculated by a corresponding model (for example, a neural network model).

According to embodiments of the present disclosure, a specific wave function model may be any appropriate model, such as a neural network-based model, etc., which may also be referred to as a specific wave function. In this way, the specific function model can derive an output characterizing the physical states based on the input physical attribute information, which can also be called wave function output/wave function value. In some embodiments, the model may be a conventional neural network-based model suitable for the molecular system, which may be referred to as a molecular neural network model, a molecular network model, or a molecular network hereinafter, such a molecular neural network model may be a wave function model that cannot reflect the physical properties of the solid system and/or meet the requirements of the wave function of the solid system, such as a model that cannot reflect periodicity, a model that cannot obtain a complex-valued output, etc., and the output obtained thereby cannot be effectively adapted to research/analysis of the solid system, however, the solution of the present disclosure can perform improved data processing based on the molecular neural network model to obtain wave function values used to characterize the microscopic system states of the solid system.

Generally speaking, the solid system utilizes periodically arranged atomic nuclei as the skeleton, so the wave function Ψ for electrons in the solid system also needs to satisfy periodic conditions. In short, the wave function Ψ needs to satisfy

Ψ(r+L)=ΨI

Among them, r means three-dimensional coordinates of an electron, and L is any normal lattice vector, such as (a, b, c) in FIG. 1B or their integer multiple combinations.

In order to reflect periodicity into the wave function or make it satisfy periodic conditions, the present disclosure proposes periodization processing on information to be input into a specific wave function model. In particular, the information used to fit the wave function may refer to information that can be input into the specific wave function model, such as physical attribute information in the microscopic system state of the solid system as mentioned above, such as information related to the spatial distribution of electrons, such as spatial coordinates, space distance, etc., of electrons.

According to some embodiments of the present disclosure, performing periodization processing on the physical attribute information in the microscopic system state of the solid system may be to periodically expand the physical attribute information or attribute information derived therefrom into a spatial range of the solid system, especially the periodic expansion of electron attribute information in solid systems, such as the spatial distances of electrons. In some embodiments, the periodization process is especially performed based on the periodicity of the smallest repetitive cell in the solid system, for example, the periodicity of periodically arranged atomic nuclei. Therefore, by performing periodization processing on the input, periodicity can be introduced into the obtained wave function output, and an output adapted to the physical properties and requirements of the solid system can be obtained.

In some embodiments of the present disclosure, the periodized physical attribute information also needs to be further processed according to the requirements of the wave function of the solid system, such as continuity requirements. In particular, the distribution curve of physical attribute information, especially the distribution curve at the periodic boundary, needs to be smoothed to meet the continuity requirements of the wave function of the solid system. In some embodiments, the distribution curve of the periodized physical attribute information is processed such that the derivatives of the distribution curve are continuous at the periodic boundary.

In some embodiments of the present disclosure, the physical attribute information is information related to the distances of electrons in the microscopic system state of the solid system, for example, including electron spatial coordinates, and the periodization process may include: determining distance information about electrons based on spatial coordinates of the electrons; expanding the distance information about electrons periodically based on arrangement periodicity of atomic nuclei in the solid system; and smoothing a distribution curve of the expanded distance information about electrons so that derivatives of the distribution curve are continuous at the periodic boundary.

Specifically, the distance information may refer to spatial distances of electrons in the solid system, such as a spatial distance between an electron and its associated atomic nucleus. As an example, the distance information about electrons can be determined based on their coordinates in a solid system. Then, the obtained distance information about electrons can be expanded to the whole spatial range according to the periodicity of atomic nucleus distribution in the solid system. For example, the periodicity of atomic nucleus distribution can correspond to an arrangement periodicity of the smallest repetitive cell, so that the distance information about the electrons in the smallest repetitive cell can be obtained, and then such distance information can be periodically and repeatedly arranged throughout the space. The electronic distance information and the expanded electronic distance information can be represented by distribution curves, for example.

According to some embodiments of the present disclosure, the periodic expansion of the physical attribute information can be implemented by operating on a vector derived based on the physical attribute information using a matrix constructed based on a function that is periodic and has derivative continuity. In some embodiments, the physical attribute information may be electron spatial coordinate information in the microscopic state of the solid system, and a crystal lattice vector can be obtained based on the electron spatial coordinates and the normal lattice vector in the solid system, thereby achieving periodic expansion.

An example of periodization processing according to an embodiment of the present disclosure will be described below. Specifically, for an electron in the microscopic system of the solid systems, the general spatial distance is defined as follows:

$$\begin{matrix}\vert \vec{r} \vert = \sqrt{{r}_{x}^{2} + {r}_{y}^{2} + {r}_{z}^{2}} , \\ ( {r}_{x} , {r}_{v} , {r}_{z} ) = ( \vec{r} \cdot {e}_{x} , \vec{r} \cdot {e}_{v} , \vec{r} \cdot {e}_{z} )\end{matrix}$$

where rₓ,r_{y},r_{z} can be equivalent to coordinates of a three-dimensional rectangular coordinate system with the atomic nucleus as the origin, eₓ,e_{y},e_{z} are the basis vectors of the three-dimensional rectangular coordinate system, namely the three directions x, y, and z.

The wave function of a solid system has the following two requirements:
a. periodic conditions, as mentioned above, which will not be described in detail here; and
b. the derivatives of the wave function with respect to the electron coordinates must be continuous. This is because that the existing method of solving the Schrodinger equation all require the derivatives of the wave function to be continuous, and the derivative continuity is a natural requirement.

In order to meet the above two requirements, the present disclosure proposes to combine the molecular network with the following periodic distance:

$$d ( \vec{r} ) = \frac{\sqrt{{AMA}^{T}}}{2 π} , A = ( r \cdot {a}_{1} , r \cdot {a}_{2} , r \cdot {a}_{3} ) ,$$

d(\vec{r}) corresponds to a general spatial distance |\vec{r}|, and A corresponds to (rₓ,r_{y} r_{z}). The difference is that in solid system, eₓ,e_{y},e_{z} is replaced by the normal lattice vector a₁,a₂,a₃ of a three-dimensional solid, which are generally linearly independent but not orthogonal to each other.

The M matrix aims to make the obtained spatial distance meet both requirements of periodicity and derivative continuity, and it can be constructed based on a function which is periodic and has derivative continuity, such as sine, cosine functions, etc. As an example, the formula for the M matrix is as follows:

$${M}_{ij} = {f}^{2} ( {ω}_{i} ) {δ}_{i j} + g ( {ω}_{i} ) g ( {ω}_{j} ) ( 1 - {δ}_{i j} ) , {ω}_{i} = r \cdot {b}_{i} , i = ( 1 , 2 , 3 )$$

M is a three-dimensional matrix, corresponding to the three-dimensional space, b₁,b₂,b₃ are inverses of the normal lattice vector (a) of the solid system. Among them, i and j take values 1, 2, and 3 respectively, and when i=j, δᵢⱼ=1, otherwise, δᵢⱼ=0. By selecting f and g functions in the following specific forms, both requirements of periodicity and derivative continuity can be satisfied. The shapes off and g are similar to that of cos, sin functions in trigonometric functions, for example, as follows:

$$f ( ω ) = \vert ω \vert ( 1 - \frac{1}{4} {\vert \frac{ω}{π} \vert}^{3} ) , g ( ω ) = ω ( 1 - \frac{3}{2} \vert \frac{ω}{π} \vert + \frac{1}{2} {\vert \frac{ω}{π} \vert}^{2} )$$

The distance d generated by the above construction is the same as the ordinary distance when r is located near the origin, and periodicity can be achieved. That is to say, the physical values input into the conventional molecular network realize periodicity, so the periodicity must be reflected in the molecular network processing process, which is equivalent to processing original non-periodic numerical values by applying a wave function that meets the periodic requirements thereto, i.e., the combination of such periodic expansion processing and the conventional molecular network is equivalent to fitting a wave function that meets the periodic requirements, and the obtained result is the wave function output that meets the periodic requirements.

FIG. 3B shows a rendering of an exemplary periodic expansion according to an embodiment of the present disclosure. Among them, taking a one-dimensional case as an example, atomic nuclei are arranged in a fixed-length period, the circular points or semi-circular points in FIG. 3B indicate nuclei or atoms, the repeated solid broken lines depict the distance between electrons and their nearest nuclei, while the smoothed periodic dotted line indicates the distance after periodic expansion according to the present disclosure, and the derivatives of the distance curve are continuous at the periodic boundary in the solid system indicated by the vertical dotted line, which is a property that the solid network must satisfy. Using this periodically expanded distance as the input of the molecular network can naturally and concisely meet the periodic requirements of the solid system, especially the periodic requirements of the wave function of the solid system. In this way, by periodizing the input of the wave function model, periodic conditions can be effectively introduced into the wave function without excessive consumption of computing resources.

It should be noted that a system wave function of a solid system is in principle a complex-valued function, which in this disclosure refers to a function whose input is a real number and whose output is a complex number. Therefore, unlike a general real number neural network, the model used for solid system calculation, especially for solid system wave function calculation, such as neural network model, must involve imaginary numbers, which is also a requirement that is not found in conventional molecular networks. In view of this, embodiments of the present disclosure propose improved data processing to acquire a wave function output that meets the requirements of a complex-valued wave function based on a conventional wave function model, that is, to acquire a wave function output in a complex-valued form.

In some embodiments of the present disclosure, the wave function output in complex form can be acquired based on a specific wave function model for wave function fitting, the specific wave function model can be a conventional wave function model, such as the above-mentioned molecular network, which can be a real number neural network. In some embodiments, a complex-valued representation can be constructed based on the output of the specific wave function model to obtain a wave function output in complex form. In one implementation, the output of a real number neural network can be duplicated as real and imaginary parts, so that a complex-valued representation can be constructed therefrom.

An exemplary implementation of complex-valued representation construction according to embodiments of the present disclosure will be described below.

A conventional molecular network will output a square matrix of molecular orbitals at the end of the network. In order to meet the complex number requirements, the original matrix output at the end of the molecular network can be doubled to be used for simulation of the real part and imaginary part of the wave function respectively, as follows.

$$( \begin{matrix}{ϕ}_{11} & … & {ϕ}_{1 N} \\ ⋮ & ⋱ & ⋮ \\ {ϕ}_{N 1} & … & {ϕ}_{NN}\end{matrix} ) \to ( \begin{matrix}{ϕ}_{11}^{R e} & … & {ϕ}_{1 N}^{R e} \\ ⋮ & ⋱ & ⋮ \\ {ϕ}_{N 1}^{R e} & … & {ϕ}_{NN}^{R e}\end{matrix} ) + i ( \begin{matrix}{ϕ}_{11}^{I m} & … & {ϕ}_{1 N}^{I m} \\ ⋮ & ⋱ & ⋮ \\ {ϕ}_{N 1}^{I m} & … & {ϕ}_{NN}^{I m}\end{matrix} )$$

The elements in the above matrix represent a series of orbitals that can be occupied by electrons in the solid system, and the value of the determinant of the matrix is the wave function value of the corresponding system. The left side of the formula represents the matrix output by a conventional molecular network, which is usually a real number output matrix. The right side represents the constructed complex form, including real and imaginary parts, which can represent the complex wave function output of a solid system. This may be equivalent to the output obtained by a wave function that satisfies the complex-valued requirements of the wave function of the solid system, and in embodiments of the present disclosure, such an output can be obtained based only on conventional wave function models, especially real wave functions, so that the processing is cost efficient and computing resources are saved.

In other embodiments of the present disclosure, physical attribute information, such as information about spatial distribution of electrons, in the microscopic system state of the solid system may be further processed to facilitate the construction of a complex-valued representation. In particular, a phase factor characterizing the microscopic system of the solid system can be applied to the physical attribute information in the microsystem state of the solid system, and a complex-valued representation including the real part and the imaginary part can also be obtained, as shown in step S304 in FIG. 3A.

As an example, a phase factor exp(ik·r), which is important for describing/characterizing the solid system, can be introduced, where E indicates the electron coordinates, and k is a specific crystal momentum vector. This phase factor originates from the famous Bloch theorem in solid system research: the electronic wave function in a solid system usually needs to be modulated by the phase factor, so introducing this phase factor can further appropriately characterize/fit the wave function of the solid system. In the calculation of the present disclosure, k can be specified in advance by calculation methods known in the art, and will not be described in detail here.

In still other embodiments of the present disclosure, the complex-valued representation generated based on applying a phase factor to physical attribute information may be combined with the complex-valued representation created based on the output of the specific wave function model. Therefore, the output obtained by combining the above two can finally be similar to the output of the solid network wave function. As a result, the complex representation of the wave function can be effectively obtained, thereby fitting of the complex-valued wave function that characterizes the solid system can be implemented effectively and accurately.

Of course, it should be noted that even if the operation of generating a complex-valued representation based on applying a phase factor to the physical attribute information indicated in step S304 above is not performed, the wave function obtained according to the embodiment of the present disclosure is still a complex-valued function, which, compared with the only real-number wave function obtained by the molecular network, can be more appropriately applied to the solid-state system to facilitate the analysis of solid-state system. Therefore, the above step S304 can be indicated by a dotted line to indicate that this step is not necessary. In addition, the above step 304 may also be included in step S303.

FIG. 3C shows an overall conceptual diagram of data processing for a solid state system according to an embodiment of the present disclosure, which shows, for the physical attribute information in the microscopic system state of the solid system, how to generate the wave function output that reflects the physical properties of solid system and/or meets the requirements of wave function of the solid system according to the embodiment of the present disclosure.

Among them, the physical attribute information in the microscopic system state of the solid system can include the electron coordinates in the microscopic system state of the solid system, the upper left part in FIG. 3C can correspond to the periodization processing of the electron coordinates, which can be implemented as described above. In particular, periodization processing is performed by using a periodic metric matrix, which can be like the matrix M as mentioned above. Such periodized information can then be input into a specific wave function model, such as a conventional molecular neural network, and the output of the wave function model is then processed to create a complex-valued representation, as shown in the upper right part of FIG. 3C.

Furthermore, the lower part in FIG. 3C may correspond to the further processing of the physical attribute information in the microscopic system state of the solid system, which can be performed using the phase factor as described above, in particular, first multiplying the electron coordinate vector with the crystal momentum vector, such as vector multiplication, dot product, etc., and then introducing a phase factor.

Finally, the complex-valued representation obtained in the upper right part of FIG. 3C is combined with the complex-valued representation obtained in the lower part of FIG. 3C by introducing the phase factor, thereby obtaining an accurate wave function output that reflects the physical properties of solid system and/or meets the requirements of wave function of the solid system.

In particular, in embodiments of the present disclosure, by being based on a conventional wave function model, such as a molecular neural network, and replacing the original distance input of the molecular network with a periodic distance, the conventional wave function model can be naturally extended to the solid system, while the computational accuracies of such models in molecular systems can be maintained, avoiding additional computational burden caused by periodic requirements.

Additionally, in embodiments of the present disclosure, complex-valued representations are generated by processing output data from the conventional wave function model and optionally by applying phase factors to physical attributes in the microscopic system state of the solid system, as a result, a wave function output that meets the complex-valued requirements can be obtained while maintaining or even improving efficiency, thereby obtaining a more accurate wave function output suitable for solid systems while taking both efficiency and accuracy into consideration. In particular, the output of the molecular network output is doubled to be used for simulation of the real part and imaginary part of the wave function respectively, in combination with the phase factor in physical theory, the complex-valued problem of the wave function can be solved and an efficient fitting of the complex-valued wave function can be achieved.

In this way, in a sense, combination of improved input and output data processing (e.g., periodization processing, complex-valued representation creation, processing of applying phase factors, etc.) according to embodiments of the present disclosure can be considered equivalent to constructing/computing a wave function that characterizes the microscopic system of the physical solid system, for example, a wave function that reflects the physical properties of the solid system and/or meets the wave function requirements of the solid system. That is, the above-described data processing according to the present disclosure may be equivalent to applying the physical attribute information of the solid system to such constructed/calculated wave function to obtain a wave function output that reflects the physical properties of the solid system and/or meets the wave function requirements of the solid system. Therefore, the calculation accuracy of the wave function model in the molecular system can be maintained, while the additional computational burden caused by periodic requirements, complex value requirements, etc. can be effectively avoided.

FIG. 3D shows a block diagram of a data processing apparatus of a solid-state system according to an embodiment of the present disclosure. The data processing apparatus 400 may include a periodization processing unit 401 configured to perform periodization processing on physical attribute information in a microscopic system state of the solid system; a model application unit 402 configured to apply the periodized physical attribute information to a specific wave function model; and a complex-valued representation creation unit 403 configured to create a complex-valued representation based on the output of the specific wave function model, to obtain wave function output in complex form. Such a wave function output in complex form reflects the physical properties of the solid system and/or meets the requirements of the wave function of the solid system, thereby being suitable for research/analysis of the solid system. It should be noted that the model application unit 402 may be the specific wave function model itself.

In some embodiments, the periodization processing unit 401 may be further configured to periodically expand the physical attribute information or information derived from the physical attribute information based on the arrangement periodicity of the atomic nuclei in the microscopic system state of the solid system, and smooth an information distribution curve obtained by periodic expansion to realize derivative continuity at boundary.

In some embodiments, the periodization processing unit 401 is further configured to perform operation on a vector derived from the physical attribute information by utilizing a matrix constructed based on a function that is periodic and has derivative continuity, to implement the periodic expansion of the physical attribute information.

In some embodiments, the physical attribute information includes electron spatial coordinates, and the periodization processing unit 401 is further configured to determine distance information about electrons based on the electron spatial coordinates; periodically expand the distance information about electrons based on the arrangement periodicity of the atomic nuclei in the solid system, and smooth a distribution curve of the distance information about electrons obtained by periodic expansion to realize derivative continuity at boundary.

In some embodiments, the complex-valued representation creation unit 403 is further configured to make the model output as a real part and an imaginary part of the complex-valued representation respectively.

In some embodiments, the data processing apparatus may further include a unit configured to apply a phase factor characterizing the microscopic system of the solid system to the electron attribute information in the solid system, and a unit configured to combine a result of applying the phase factor to the electron attribute information with a complex-valued representation. It should be noted that such two units can also be combined into one unit to achieve the above functions. In some exemplary implementations, such two units may be incorporated into other units in the data processing apparatus, in particular the complex-valued representation creation unit. As an example, the complex-valued representation creation unit 403 may also be further configured to apply a phase factor characterizing the microscopic system of the solid system to the electron attribute information in the solid system, and combine a result of applying the phase factor to the electron attribute information with a complex-valued representation.

It should be noted that the above-mentioned various units are merely logical modules divided according to specific functions implemented by the units, and are not used to limit specific implementations. For example, the units may be implemented by software, hardware, or a combination of software and hardware. In actual implementation, the above-mentioned various units may be implemented as separate physical entities, or may be implemented by a single entity (for example, a processor (a CPU, a DSP, etc.), or an integrated circuit). In addition, the above-mentioned various units are shown with dotted lines in the drawings to indicate that these units may not actually exist, and the operations/functions implemented by them may be implemented by a processing circuit.

In addition, although not shown, the apparatus may further include a memory, which may store various information generated during the operations by the apparatus and the various units included in the apparatus, program and data used for the operations, data to be sent by a communication unit, etc. The memory may be a volatile memory and/or a non-volatile memory. For example, the memory may include, but is not limited to, a random-access memory (RAM), a dynamic random-access memory (DRAM), a static random-access memory (SRAM), a read-only memory (ROM), and a flash memory. Certainly, the memory may alternatively be located outside of the apparatus. Optionally, although not shown, the apparatus may further include a communication unit, which may be used to communicate with other apparatuses. In an example, the communication unit may be implemented in an appropriate manner known in the art, for example, including communication components such as an antenna array and/or a radio frequency link, various types of interfaces, communication units, and the like. This is not described in detail here. In addition, the apparatus may further include other components not shown, such as a radio frequency link, a baseband processing unit, a network interface, a processor, and a controller. This will not be described in detail here.

A solid system analysis scheme according to the present disclosure will be described below. In embodiments of the present disclosure, for a specific solid system, the wave function of the solid system fitted according to the embodiments of the present disclosure can be used to solve a corresponding Schrodinger equation characterizing the solid system, thereby realizing accurate research/analysis of the solid system to effectively and accurately acquire the physical properties of the solid system. Solving the Schrodinger equation can be performed in various ways known in the art and thus will not be described in detail here.

FIG. 3E shows a flow chart of an analysis method for a solid system according to an embodiment of the present disclosure, where the solid system analysis mainly involves analysis of various appropriate physical properties of the solid system, especially physical properties related to energy, and the like.

In the method 310, in step S311, the data processing method according to the embodiment of the present disclosure may be applied to acquire an output that reflects the physical properties of the solid system and/or meets the wave function requirements of the solid system. The output here may correspond to the previously described results obtained by the data processing method or data processing apparatus according to embodiments of the present disclosure, such as results of complex-valued representations or further combinations thereof.

In step S312, the output is applied to solve a specific equation characterizing the microscopic system of the solid system to determine the physical properties of the solid system. In particular, the specific equation characterizing the microscopic system of the solid system is a Schrodinger equation describing the microscopic system, and the physical properties of the solid system are properties related to energy distribution of the solid system.

FIG. 3F shows a block diagram of an analysis apparatus for a solid system according to an embodiment of the present disclosure. The analysis apparatus 410 may include an acquisition unit 411 configured to apply the data processing method according to an embodiment of the present disclosure to acquire an output that reflects physical properties of the solid system and/or meets wave function requirements of the solid system; and a solving unit 412 configured to apply the output to solve the specific equations characterizing the microscopic system of the solid system, to determine the physical properties of the solid system.

It should be noted that the analysis apparatus for a solid system and its units here can be implemented in various appropriate ways, for example, it can be implemented in manners similar to that for implementation of the data processing apparatus and its units as above, and will not be described in detail here.

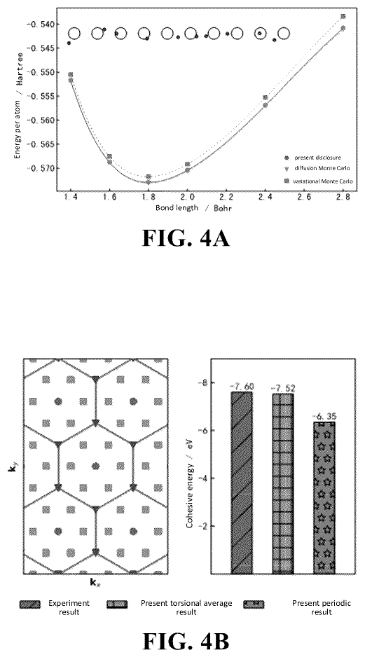

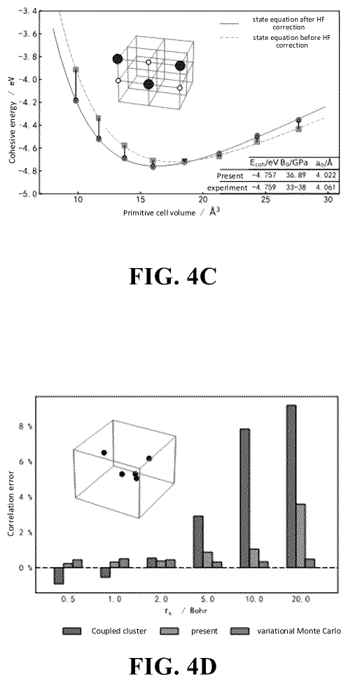

As an example, embodiments of the present disclosure performs test in several classical solid systems, and perform comparison with the results and experimental data of mature methods in the field. The solid system includes but is not limited to one-dimensional hydrogen chain, two-dimensional graphene, three-dimensional lithiated hydrogen, uniform electron gas, etc., and the results obtained by applying embodiments of the present disclosure are shown in FIGS. 4A to 4D.

FIG. 4A shows the results when the solid system is a one-dimensional hydrogen chain, the figure shows the energy of each H atom in the hydrogen chain relative to the bond length, it can be seen that the calculation result of the present disclosure is substantially consistent with the existing methods, such as high-precision diffused Monte Carlo method, and superior to other variational Monte Carlo methods.

FIG. 4B shows the results when the solid system is two-dimensional graphene, the figure shows the cohesive energy of the graphene in a histogram, it can be seen that the calculation result of the present disclosure is substantially consistent with the experimental results.

FIG. 4C shows the results when the solid system is three-dimensional lithiated hydrogen, the figure shows the cohesive energy relative to the original cell volume, it can be seen that the calculation result of the present disclosure is substantially consistent with the experimental results.

FIG. 4D shows the results when the solid system is a uniform electron gas, in the figure, the relevant errors are shown in a histogram, it can be seen that the calculation result of the present disclosure is substantially consistent with, or even better than, the calculation results of other high-precision methods.

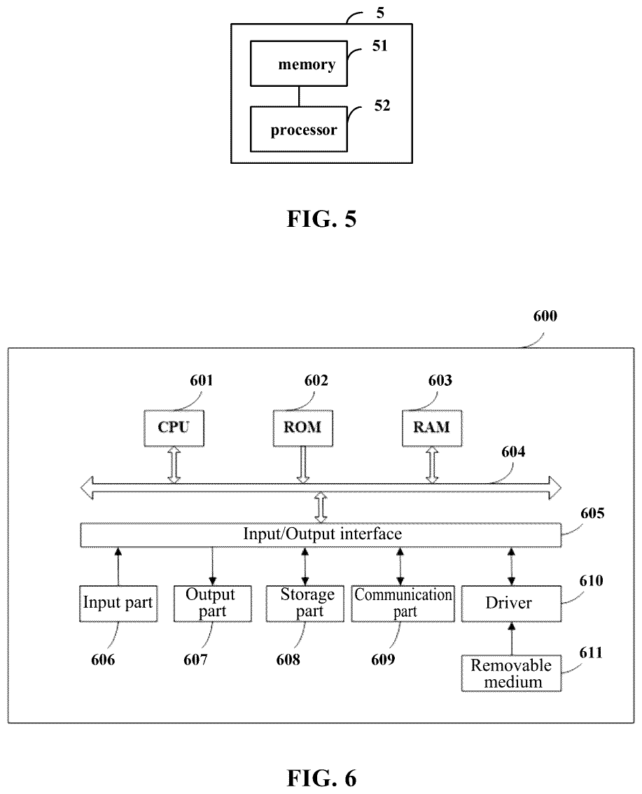

Some embodiments of the present disclosure further provide an electronic device that can be operable to implement the operations/functions of the above-mentioned model pre-training device and/or model training device. FIG. 5 is a block diagram of an electronic device according to some embodiments of the present disclosure. For example, in some embodiments, the electronic device 5 may be various types of devices, for example, may include, but not limited to, mobile terminals such as a mobile phone, a notebook computer, a digital broadcast receiver, a personal digital assistant (PDA), a tablet computer (PAD), a portable multimedia player (PMP), and a vehicle-mounted terminal (such as a vehicle navigation terminal), and fixed terminals such as a digital TV and a desktop computer. For example, the electronic device 5 may include a display panel configured to display data used in the solution according to the present disclosure and/or execution results. For example, the display panel may be in various shapes, such as a rectangular panel, an elliptical panel, or a polygonal panel, etc. In addition, the display panel may be a flat panel, a curved panel, or even a spherical panel.

As shown in FIG. 5, the electronic device 5 in this embodiment includes: a memory 51 and a processor 52 coupled to the memory 51. It should be noted that the components of the electronic device 50 shown in FIG. 5 are merely exemplary and non-limiting. The electronic device 50 may further have other components according to actual application requirements. The processor 52 may control other components in the electronic device 5 to perform desired functions.

In some embodiments, the memory 51 is configured to store one or more computer-readable instructions. The processor 52 is configured to run computer-readable instructions, and the computer-readable instructions, when run by the processor 52, cause the method according to any one of the above embodiments to be implemented. For specific implementations of the steps of the method and related explanation content, reference may be made to the above embodiments, and repetitions will not be repeated here.

For example, the processor 52 and the memory 51 may communicate with each other directly or indirectly. For example, the processor 52 and the memory 51 may communicate with each other via a network. The network may include a wireless network, a wired network, and/or any combination of wireless networks and wired networks. The processor 52 and the memory 51 may also communicate with each other through a system bus, which is not limited in the present disclosure.

For example, the processor 52 may be embodied as various appropriate processors, processing apparatuses, etc., such as a central processing unit (CPU), a graphics processing unit (GPU), and a network processor (NP); or may be a digital signal processor (DSP), an application-specific integrated circuit (ASIC), a field programmable gate array (FPGA) or other programmable logic devices, a discrete gate or transistor logic device, or a discrete hardware component. The central processing unit (CPU) may be of an X86 or ARM architecture, etc. For example, the memory 51 may include any combination of various forms of computer-readable storage media, for example, a volatile memory and/or a non-volatile memory. The memory 51 may include, for example, a system memory, and the system memory stores, for example, an operating system, an application, a boot loader, a database, and other programs. Various applications and various data may also be stored in the storage medium.

In addition, according to some embodiments of the present disclosure, when various operations/processing according to the present disclosure are implemented by software and/or firmware, programs constituting the software may be installed from the storage medium or a network to a computer system with a dedicated hardware structure, such as a computer system 600 shown in FIG. 6. When installed with various programs, the computer system can perform various functions, including the functions described above, etc. FIG. 6 is a block diagram of an example structure of a computer system that can be used according to an embodiment of the present disclosure.

In FIG. 6, a central processing unit (CPU) 601 performs various processing according to a program stored in a read-only memory (ROM) 602 or loaded from a storage part 608 into a random-access memory (RAM) 603. Data required for the CPU 601 to perform various processing and the like is also stored in the RAM 603 as required. The central processing unit is merely exemplary, and it may alternatively be other types of processors, such as the various processors described above. The ROM 602, the RAM 603, and the storage part 608 may be various forms of computer-readable storage media, as described below. It should be noted that although the ROM 602, the RAM 603, and the storage device 608 are shown separately in FIG. 6, one or more of them may be combined or located in the same or different memories or storage modules.

The CPU 601, the ROM 602, and the RAM 603 are connected to one another through a bus 604. An input/output interface 605 is also connected to the bus 604.

The following components are connected to the input/output interface 605: an input part 606, for example, a touch screen, a touchpad, a keyboard, a mouse, an image sensor, a microphone, an accelerometer, or a gyroscope; an output part 607, including a display, such as a cathode-ray tube (CRT), a liquid crystal display (LCD), a speaker, or a vibrator; the storage part 608, including a hard disk, a magnetic tape, etc.; and a communication part 609, including a network interface card, such as a LAN card, or a modem. The communication part 609 allows communication processing to be performed via a network such as the Internet. It is easy to understand that although the various apparatuses or modules in the electronic device 600 shown in FIG. 6 communicate through the bus 604, they may alternatively communicate through a network or other means, where the network may include a wireless network, a wired network, and/or any combination of wireless networks and wired networks.

A driver 610 is also connected to the input/output interface 605 as required. A removable medium 611 such as a magnetic disk, an optical disk, a magneto-optical disk, or a semiconductor memory, is installed on the driver 610 as required, such that a computer program read therefrom is installed into the storage part 608 as required.

When the above-described series of processing is implemented by software, programs constituting the software may be installed from a network such as the Internet or a storage medium such as the removable medium 611.

According to an embodiment of the present disclosure, the process described above with reference to the flowcharts may be implemented as a computer software program. For example, this embodiment of the present disclosure includes a computer program product, which includes a computer program carried on a computer-readable medium, where the computer program includes program code for performing the method according to the embodiments of the present disclosure. In such an embodiment, the computer program may be downloaded from a network through the communication device 609 and installed, installed from the storage device 608, or installed from the ROM 602. When the computer program is executed by the CPU 601, the above-mentioned functions defined in the method of the embodiments of the present disclosure are performed.

It should be noted that, in the context of the present disclosure, the computer-readable medium may be a tangible medium that may contain or store a program for use by or in conjunction with an instruction execution system, apparatus, or device. The computer-readable medium may be a computer-readable signal medium, a computer-readable storage medium, or any combination thereof. The computer-readable storage medium may be, for example, but is not limited to, an electric, magnetic, optical, electromagnetic, infrared, or semi-conductive system, apparatus, or device, or any combination thereof. A more specific example of the computer-readable storage medium may include, but is not limited to: an electrical connection having one or more wires, a portable computer magnetic disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof. In the present disclosure, the computer-readable storage medium may be any tangible medium containing or storing a program which may be used by or in combination with an instruction execution system, apparatus, or device. In the present disclosure, the computer-readable signal medium may include a data signal propagated in a baseband or as a part of a carrier, the data signal carrying computer-readable program code. The propagated data signal may be in various forms, including but not limited to an electromagnetic signal, an optical signal, or any suitable combination thereof. The computer-readable signal medium may also be any computer-readable medium other than the computer-readable storage medium. The computer-readable signal medium can send, propagate, or transmit a program used by or in combination with an instruction execution system, apparatus, or device. The program code contained in the computer-readable medium may be transmitted by any suitable medium, including but not limited to: electric wires, optical cables, radio frequency (RF), etc., or any suitable combination thereof.

The above computer-readable medium may be contained in the above electronic device. Alternatively, the computer-readable medium may exist independently, without being assembled into the electronic device.

In some embodiments, there is further provided a computer program. The computer program includes instructions that, when executed by a processor, cause the processor to perform the method in any one of the above embodiments. For example, the instructions may be embodied as computer program code.

In the embodiments of the present disclosure, the computer program code for performing the operations of the present disclosure may be written in one or more programming languages or a combination thereof, where the programming languages include, but are not limited to, an object-oriented programming language, such as Java, Smalltalk, and C++, and further include conventional procedural programming languages, such as “C” language or similar programming languages. The program code may be completely executed on a computer of a user, partially executed on a computer of a user, executed as an independent software package, partially executed on a computer of a user and partially executed on a remote computer, or completely executed on a remote computer or server. In the case of the remote computer, the remote computer may be connected to the computer of the user via any kind of network, including a local area network (LAN) or a wide area network (WAN), or may be connected to an external computer (for example, connected via the Internet with the aid of an Internet service provider).

The flowcharts and block diagrams in the accompanying drawings illustrate the possibly implemented architecture, functions, and operations of the system, method, and computer program product according to various embodiments of the present disclosure. In this regard, each block in the flowchart or block diagram may represent a module, program segment, or part of code, and the module, program segment, or part of code contains one or more executable instructions for implementing the specified logical functions. It should also be noted that, in some alternative implementations, the functions marked in the blocks may also occur in an order different from that marked in the accompanying drawings. For example, two blocks shown in succession can actually be performed substantially in parallel, or they can sometimes be performed in the reverse order, depending on the functions involved. It should also be noted that each block in the block diagram and/or the flowchart, and a combination of the blocks in the block diagram and/or the flowchart may be implemented by a dedicated hardware-based system that executes specified functions or operations, or may be implemented by a combination of dedicated hardware and computer instructions.

The related modules, components, or units described in the embodiments of the present disclosure may be implemented by software, or may be implemented by hardware. The names of the modules, components, or units do not constitute a limitation on the modules, components, or units themselves in some cases.

The functions described herein above may be performed at least partially by one or more hardware logic components. For example, without limitation, exemplary hardware logic components that may be used include: a field programmable gate array (FPGA), an application-specific integrated circuit (ASIC), an application-specific standard product (ASSP), a system on a chip (SOC), a complex programmable logic device (CPLD), and the like.

The present disclosure may be implemented in any form described here, including but not limited to the example embodiments enumerated below, which describe structures, features, and functions of some parts of the embodiments of the present invention.

According to some embodiments of the present disclosure, there is provided a data processing method for a solid system. The method includes the following steps: performing periodization processing on physical attribute information in a microscopic system state of the solid system; applying the periodized physical attribute information to a specific wave function model to obtain output of the specific wave function model; and creating a complex-valued representation based on the output of the specific wave function model.

According to some embodiments of the present disclosure, the physical attribute information may include information related to spatial distribution of electrons in the solid system, and the specific wave function model is a wave function model that characterizes the state of electrons in the solid system.

According to some embodiments of the present disclosure, the periodization process may include: periodically expanding the physical attribute information or information derived from the physical attribute information based on the arrangement periodicity of the atomic nuclei in the microscopic system state of the solid system, and smoothing an information distribution curve obtained by periodic expansion to realize derivative continuity at boundary.

According to some embodiments of the present disclosure, the periodization process may include performing operation on a vector derived from the physical attribute information by utilizing a matrix constructed based on a function that is periodic and has derivative continuity, to implement the periodic expansion of the physical attribute information.

According to some embodiments of the present disclosure, the physical attribute information may include electron spatial coordinates in the microscopic system state of the solid system, and the periodization process may include: determining distance information about electrons based on the electron spatial coordinates; periodically expanding the distance information about electrons based on the arrangement periodicity of the atomic nuclei in the solid system, and smoothing a distribution curve of the distance information about electrons obtained by periodic expansion to realize derivative continuity at boundary.

According to some embodiments of the present disclosure, creating the complex-valued representation may include duplicating the output of the specific wave function model as real and imaginary parts of the complex-valued representation respectively.

According to some embodiments of the present disclosure, the method may further include: applying a phase factor characterizing the microscopic system of the solid system to the physical attribute information in a microscopic system state of the solid system, and combining a result of applying the phase factor to the physical attribute information with a complex-valued representation.

According to some embodiments of the present disclosure, the physical attribute information may include electron spatial coordinates in a microscopic system state of the solid system, and the phase factor is exp (lk·\vec{r}), wherein \vec{r} are the electron coordinates, and k is a specific crystal momentum vector.

According to some embodiments of the present disclosure, the specific wave function model may be a molecular neural network wave function model.

According to some embodiments of the present disclosure, an analysis method for a solid system is provided. The method includes the following steps: applying the data processing method according to any embodiment of the present disclosure to acquire an output that reflects physical properties of the solid system and/or meets the wave function requirements of the solid system; and applying said output that meets the wave function requirements of the solid system to solve a specific equation characterizing the microscopic system of the solid system to determine the physical properties of the solid system.

According to some embodiments of the present disclosure, the specific equation characterizing the microscopic system of the solid system may be a Schrodinger equation describing the microscopic system, and the physical properties of the solid system are properties related to energy distribution of the solid system.

According to some embodiments of the present disclosure, a data processing apparatus for a solid system is provided, the apparatus comprising: a periodization processing unit configured to perform periodization processing on physical attribute information in a microscopic system state of the solid system; a model application unit configured to apply the periodized physical attribute information to a specific wave function model; and a complex-valued representation creation unit configured to create a complex-valued representation based on an output of the specific wave function model.

According to some embodiments of the present disclosure, an analysis apparatus for a solid system is provided, the apparatus includes: an acquisition unit configured to apply the method according to any embodiment of the present disclosure to acquire an output that reflects physical properties of the solid system and/or meets the wave function requirements of the solid system; and a solving unit configured to apply said output that meets the wave function requirements of the solid system to solve a specific equation characterizing the microscopic system of the solid system to determine the physical properties of the solid system.

According to some other embodiments of the present disclosure, there is provided an electronic device, which may include: a memory; and a processor coupled with the memory, wherein the memory has instructions therein, and wherein the instructions, when executed by the processor, cause the electronic device to execute the method according to any one of the embodiments of the present disclosure.

According to some other embodiments of the present disclosure, there is provided a computer-readable storage medium having a computer program stored thereon, wherein the program, when executed by a processor, causes the method according to any one of the embodiments of the present disclosure to be implemented.

According to some other embodiments of the present disclosure, there is provided a computer program including instructions that, when executed by a processor, cause the method according to any one of the embodiments of the present disclosure to be implemented.

According to some other embodiments of the present disclosure, there is provided a computer program product including instructions that, when executed by a processor, cause the method according to any one of the embodiments of the present disclosure to be implemented.

The foregoing descriptions are merely some embodiments of the present disclosure and explanations of the applied technical principles. Those skilled in the art should understand that the scope of disclosure involved in the present disclosure is not limited to the technical solutions formed by specific combinations of the foregoing technical features, and shall also cover other technical solutions formed by any combination of the foregoing technical features or equivalent features thereof without departing from the foregoing concept of disclosure. For example, a technical solution formed by a replacement of the foregoing features with technical features with similar functions disclosed in the present disclosure (but not limited thereto) also falls within the scope of the present disclosure.

In the description provided herein, numerous specific details are set forth. However, it is understood that the embodiments of the present invention may be practiced without these specific details. In other cases, well-known methods, structures, and techniques have not been shown in detail in order not to obscure the understanding of this description.

In addition, although the various operations are depicted in a specific order, it should be understood as requiring these operations to be performed in the specific order shown or in a sequential order. Under certain circumstances, multitasking and parallel processing may be advantageous. Similarly, although several specific implementation details are included in the foregoing discussions, these details should not be construed as limiting the scope of the present disclosure. Some features that are described in the context of separate embodiments can also be implemented in combination in a single embodiment. In contrast, various features described in the context of a single embodiment may alternatively be implemented in a plurality of embodiments individually or in any suitable sub combination.

While some specific embodiments of the present disclosure have been exemplarily described in detail, it should be understood by those skilled in the art that the above examples are merely for illustration and are not intended to limit the scope of the present disclosure. Those skilled in the art should understand that various modifications can be made to the above embodiments, without departing from the scope and spirit of the present disclosure. The scope of the present disclosure is defined by the appended claims.

What is claimed is:

1. A data processing method for a solid system, the method comprises:
performing periodization processing on physical attribute information in a microscopic system state of the solid system;
applying the periodized physical attribute information to a specific wave function model; and
creating a complex-valued representation based on the output of the specific wave function model.

2. The method of claim 1, wherein, the physical attribute information comprises information related to spatial distribution of electrons in the solid system, and the specific wave function model is a wave function model that characterizes the state of electrons in the solid system.

3. The method of claim 1, wherein, the periodization process comprises:
periodically expanding the physical attribute information based on the arrangement periodicity of the atomic nuclei in the microscopic system state of the solid system, and
smoothing an information distribution curve obtained by periodic expansion to realize derivative continuity at boundary.

4. The method of claim 1, wherein, the periodization process comprises:
performing operation on a vector derived from the physical attribute information by utilizing a matrix constructed based on a function that is periodic and has derivative continuity, to implement the periodic expansion of the physical attribute information.

5. The method of claim 1, wherein, the physical attribute information comprises electron spatial coordinates in the microscopic system state of the solid system, and the periodization process comprises:
determining distance information about electrons based on the electron spatial coordinates;
periodically expanding the distance information about electrons based on the arrangement periodicity of the atomic nuclei in the solid system, and
smoothing a distribution curve of the distance information about electrons obtained by periodic expansion to realize derivative continuity at boundary.

6. The method of claim 1, wherein, the creating the complex-valued representation comprises duplicating the output of the specific wave function model as real and imaginary parts of the complex-valued representation respectively.

7. The method of claim 1, wherein, the method further comprises:
applying a phase factor characterizing the microscopic system of the solid system to the physical attribute information in a microscopic system state of the solid system, and
combining a result of applying the phase factor to the physical attribute information with a complex-valued representation.

8. The method of claim 7, wherein, the physical attribute information may include electron spatial coordinates in a microscopic system state of the solid system, and the phase factor is exp(ik·\vec{r}), wherein \vec{r} are the electron coordinates, and k is a specific crystal momentum vector.

9. The method of claim 1, wherein, the specific wave function model may be a molecular neural network wave function model.

10. The method of claim 1, wherein,
an output that reflects physical properties of the solid system and/or meets the wave function requirements of the solid system is acquired; and
wherein said output is applied to solve a specific equation characterizing the microscopic system of the solid system to determine the physical properties of the solid system.

11. The method of claim 10, wherein, the specific equation characterizing the microscopic system of the solid system is a Schrodinger equation describing the microscopic system, and the physical properties of the solid system are properties related to energy distribution of the solid system.

12-13. (canceled)

14. An electronic device, comprises:
a memory; and
a processor coupled with the memory, wherein the memory has executable instructions therein, and wherein the executable instructions, when executed by the processor, cause the electronic device to execute a data processing method for a solid system, the method comprising:
performing periodization processing on physical attribute information in a microscopic system state of the solid system;
applying the periodized physical attribute information to a specific wave function model; and
creating a complex-valued representation based on the output of the specific wave function model.

15. The electronic device of claim 14, wherein, the executable instructions, when executed by the processor, cause the electronic device to perform the periodization processing, comprising:
periodically expanding the physical attribute information based on the arrangement periodicity of the atomic nuclei in the microscopic system state of the solid system, and smoothing an information distribution curve obtained by periodic expansion to realize derivative continuity at boundary, and/or
wherein, the executable instructions, when executed by the processor, cause the electronic device to perform the periodization processing, comprising:
performing operation on a vector derived from the physical attribute information by utilizing a matrix constructed based on a function that is periodic and has derivative continuity, to implement the periodic expansion of the physical attribute information, and/or
wherein, the physical attribute information comprises electron spatial coordinates in the microscopic system state of the solid system, and wherein, the executable instructions, when executed by the processor, cause the electronic device to perform the periodization processing, comprising:
determining distance information about electrons based on the electron spatial coordinates;
periodically expanding the distance information about electrons based on the arrangement periodicity of the atomic nuclei in the solid system, and
smoothing a distribution curve of the distance information about electrons obtained by periodic expansion to realize derivative continuity at boundary.

16. The electronic device of claim 14, wherein, the executable instructions, when executed by the processor, cause the electronic device to perform the following:
applying a phase factor characterizing the microscopic system of the solid system to the physical attribute information in a microscopic system state of the solid system, and
combining a result of applying the phase factor to the physical attribute information with a complex-valued representation.

17. The electronic device of claim 14,
wherein an output that reflects physical properties of the solid system and/or meets the wave function requirements of the solid system is acquired; and
wherein, the executable instructions, when executed by the processor, cause the electronic device to perform the following:
applying said output to solve a specific equation characterizing the microscopic system of the solid system to determine the physical properties of the solid system.

18. A non-transitory computer-readable storage medium having executable instructions stored thereon, wherein the executable instructions, when executed by a processor, causes the processor to implement:
performing periodization processing on physical attribute information in a microscopic system state of the solid system;
applying the Periodized physical attribute information to a specific wave function model; and
creating a complex-valued representation based on the output of the specific wave function model.

19-20. (canceled)

21. The non-transitory computer-readable storage medium of claim 18, wherein, the executable instructions, when executed by the processor, cause the processor to implement:
periodically expanding the physical attribute information based on the arrangement periodicity of the atomic nuclei in the microscopic system state of the solid system, and smoothing an information distribution curve obtained by periodic expansion to realize derivative continuity at boundary, and/or
wherein, the executable instructions, when executed by the processor, cause the processor to implement:
performing operation on a vector derived from the physical attribute information by utilizing a matrix constructed based on a function that is periodic and has derivative continuity, to implement the periodic expansion of the physical attribute information, and/or
wherein, the physical attribute information comprises electron spatial coordinates in the microscopic system state of the solid system, and wherein, the executable instructions, when executed by the processor, cause the processor to implement:
determining distance information about electrons based on the electron spatial coordinates;
periodically expanding the distance information about electrons based on the arrangement periodicity of the atomic nuclei in the solid system, and
smoothing a distribution curve of the distance information about electrons obtained by periodic expansion to realize derivative continuity at boundary.

22. The non-transitory computer-readable storage medium of claim 18, wherein, the executable instructions, when executed by the processor, cause the processor to implement:
applying a phase factor characterizing the microscopic system of the solid system to the physical attribute information in a microscopic system state of the solid system, and
combining a result of applying the phase factor to the physical attribute information with a complex-valued representation.

23. The non-transitory computer-readable storage medium of claim 18, wherein an output that reflects physical properties of the solid system and/or meets the wave function requirements of the solid system is acquired; and
wherein, the executable instructions, when executed by the processor, cause the processor to implement:
applying said output to solve a specific equation characterizing the microscopic system of the solid system to determine the physical properties of the solid system.
